(19) United States
(12) Patent Application Publication
(10) Pub. No.: US 2011/0319841 A1
(43) Pub. Date: Dec. 29, 2011

(54) Device for reducing wound trauma with an absorbent pad housing

(76) Inventor: Jill Marie Romie, Gainesville, FL (US)

(21) Appl. No.: 12/817,580

(22) Filed: Jun. 17, 2010

(51) Int. Cl. A61F 7/10; A61F 13/45

(52) U.S. Cl. 604/291

(57) ABSTRACT

The device is provided to reduce trauma caused by childbirth or other traumas and absorb such fluids which would be expelled after such a trauma. In particular the device is said to reduce the trauma of the wound in the perineal region or other regions, and absorb blood and other discharges after such a trauma. The device is comprised of a cotton maternity sanitary napkin which is an absorbent housing for the instant cooling ice pack which is cooled prior to application to the perineal region. To activate the ice pack one must squeeze it and wait to hear a pop sound which allows the chemical to mix creating the cooling sensation. The said device contains an adhesive strip which can be activated by pulling off the plastic tape cover. The adhesive strip then can be placed in the woman's underwear to ensure a secure placement of the device.

The invention which is the subject of this application is a device to alleviate the condition of pain and trauma caused by open wounds particularly the perineal area after childbirth.

Pernineal and rectal trauma causes a great deal of distress and pain in women after childbirth. Pain in this area may be increased if an episiotomy was performed. Also there is increased pain if forceps or a vacuum was used to remove the baby. Pernineal trauma occurs in approximately 70% of women after child birth. Many times there was an open wound that either tore or was cut and then stitched shut. In conjunction with the perineal trauma women experience significant bleeding for an average of 6 weeks after child birth. The complication of bleeding and an open wound cause great discomfort to many new mothers.

It is well know that perineal trauma is an issue after childbirth. In the past women have been offered anything from ice cubes, to ice packs but there was never a device to treat this specific condition. Ice cubes are very uncomfortable to sit on for women and are often too cold which has the effect of numbing the area but not being comfortable or long lasting. Ice packs have also been used but they are very hard, not shaped for the area, and uncomfortable to use.

Gel packs have also been used as a compress but they are often bulky and are used mostly for sports injuries. They are not designed in order to relieve perineal and rectal trauma after childbirth

EpiFoam is a substance that has also been used to treat this area. It is an anti inflammatory steroid which acts as a pain reliever but does not relieve the pressure or have a cooling effect.

In recent time Feme Pad was created which is a long gel pad specifically designed to relive perineal and rectal pain after childbirth. While the Feme Pad does relieve pressure and have a cooling effect to the area it is unable to collect blood and fluids that are expelled after child birth. Maternity pads are often used after child birth to collect blood and bodily fluids. The Feme Pad states that it is able to be used in conjunction with a maternity pad to collect blood but doing so is very bulky and uncomfortable. In addition the Feme Pad must be frozen after they warm up and reused which is unsanitary.

It is clear that there is a need for an effective device to not only absorb fluids but provide relief to the perineal trauma by cooling the area. This device is able to combine the cooling effects desired and the absorbency needed after child birth.

In the first aspect of the invention there is to provide relief to the perineal and rectal area after trauma. The said device is an instant cooling pack which is twisted until the ingredients mix and cooling effect results of the chemical reaction. The ice pack is then sealed in a maternity pad. The ice pack has a high thermal capacity and is applied directly to the perineal area to provide a cooling effect and an occluding effect on the open wound.

The ice pack is housed in a maternity pad. This allows the device not only to provide cooling relief but also the absorbent maternity pad needed in one easy and comfortable device.

This device will provide cooling relief to the area of trauma as without delaying the normal healing process. Also the device will inhibit swelling due to the cooling effect.

The ice pack portion of the device is comprised of a plastic bag that hosts urea granules and a small bag of water. When the device is twisted the small bag of water will pop allowing the urea granules and water to mix. This mixing creates an endothermic reaction and the by product is a drop in temperature.

To activate the device one must twist the pad until the water bag breaks and then shake the pad so that the urea granules and water are mixed completely. The pad will stay cold for approximately 45 minutes. The advantage is that the ice pack does not need to be refrigerated so that it can be used anywhere without waiting for the material to cool. In addition the pad is disposable so a clean sanitary one is used each time.

The pad housing is a standard maternity size of 380 mm with 10 cm of width. This shape is designed to cover the damaged perineal and rectal areas.

In yet a further aspect of the invention there is provided a method of treating person for the relief of perineal and or rectal trauma associated with child birth by application of said device. Said device is comprised of a standard maternity pad with an ice pack insert 6 cm wide and 380 mm long. Said ice pack is activated by twisting which breaks a water bag inside the ice pack and reacts with urea granules to create a cooling effect. The ice pack is laid in the middle of the standard maternity pad then sealed in when the liquid permeable top layer is sealed shut. The device once cooled is applied directly to the perineal area by removing pads adhesive strip cover and placing adhesive strip in crotch of underpants. This provides a cooling effect, occluding effect, and is absorbent at the same time.

This invention provides a method of treating a person with an open wound by the application of a device on the open wound. Said device is comprised of an absorbent pad which holds an instant ice pack. Once activated the device cools the area as well as absorb blood and other fluids. The device has a cooling and occluding effect as well as absorbency.

### BRIEF DESCRIPTION OF THE DRAWINGS

Specific embodiments of the invention are now described with reference to the accompanying drawings, wherein:

FIG. 1: There is illustrated a device according to the invention in which in this case is shaped for relief of perineal pain. The device is an instant ice pack housed in a standard maternity pad and then sealed. The device embodiment is shown relatively long to fit comfortably in the perineal region. 1 Is the cotton pad housing. 2 Is the instant ice pack.

FIG. 2: Illustrates the bottom of the device, where there is an adhesive strip. To activate one pulls the adhesive strip cover and the adhesive area is revealed. The adhesive strip is then placed in the crotch area of an undergarment such as a panty and applied to the perineal region. 1 Is the cotton pad housing. 3 is the adhesive strip.

FIG. 3: Illustrates the cross section of said device. The instant ice pack runs the length of the maternity pad housing but is only about 1/3 the width of the pad housing, allowing for fluids to run to the sides and be absorbed in the cotton of the maternity pad. The instant ice pack is running down the middle of the pad housing where it can provide cooling relief to the perineal and rectal area after a trauma such as child birth. 1 is the cotton pad housing. 2 is the instant ice pack. 3 is the adhesive stip.

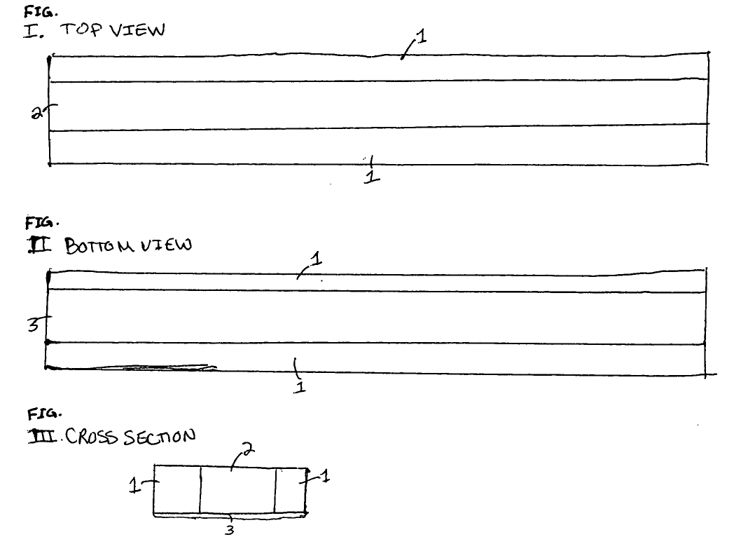

What is claimed is:

1. A device for the relief of perineal trauma said device is comprised of an instant ice pack housed in a maternity pad and sealed. The instant ice pack is composed of a plastic bag which holds urea granules and a smaller plastic bag filled with antimicrobial treated water. To activate said ice pack one must squeeze the ice pack causing the gab of water to break. The water then mixes with the urea granules and creates an exothermic by product of coolness. Said device is configured for direct application to the perineal region and cools the affected area and has an occluding effect on the open wound. In addition the device has a cushioning effect and is absorbent to hold blood and other fluids that are expelled.

2. A device according to claim 1 wherein the instant ice pack is made of urea granules and a bag of treated water

3. A device according to claim 1 wherein the instant ice pack is housed in an absorbent maternity pad.

4. A device according to claim 1, wherein the elongated form is made to fit the perineal region.

5. A device according to claim 4, wherein the device is about 10 cm wide by 288 mm long and 3 cm thick

6. A device according to claim 1, wherein the maternity pad housing has an adhesive strip which is used to secure the device to underwear.

7. A device according to claim 1. Wherein the device is activated by squeezing the ice pack to pop the water bag and start the reaction of the urea granules and water to produce a cooling effect

8. A device according to claim 1 wherein in use the device is applied to the perineal area in a cooled condition and heats up gradually thereafter. Once the device is no longer cooled or filled with blood or fluid the device is disposable and there for a new sanitary device is applied.

9. A device comprising of a housing which is a standard maternity pad with a adhesive strip on the bottom a water tight baffle bottom layer, absorbing cotton and a top water permeable layer. The instant ice pack is placed on top of the cotton layer and under the top water permeable layer. The instant ice pack is 1/3 the width of the maternity pad housing so that fluids and blood maybe collected while still providing cooling relief to the perineal area.

10. A device according to claim 9, wherein said device also has an added effect of cushioning the open would and applying pressure to the area surrounding said wound.

11. A device according to claim 1, wherein there is a cooling effect to the perineal area as well as an occluding effect to an open wound.

12. A device for the relief of perineal and or rectal trauma said device comprising:
A. A housing of a standard maternity sanitary napkin with an adhesive strip, a baffle layer, a cotton absorbent layer, and a top liquid Permeable layer. This housing is absorbent to blood and bodily fluids expelled.
B. An instant ice pack sealed within the maternity pad housing above the cotton and below the liquid permeable layer comprised urea granules and a treated bag of water inside a plastic bag. The ice pack is squeezed and the chemical reaction ensues when the water bag is broken and it mixes with the urea granules. In a cooled state to conform to the perineal area the said device provides cooling relief to the traumatized area, an occluding effect to any open wounds and a cushioning effect to the area while being disposable, portable, and absorbent.
